(19) United States
(12) Patent Application Publication
(10) Pub. No.: US 2019/0119678 A1
(43) Pub. Date: Apr. 25, 2019

(54) MEANS AND METHODS FOR INACTIVATING THERAPEUTIC DNA IN A CELL

(71) Applicant: Ruprecht-Karls-Universität Heidelberg, Heidelberg (DE)

(72) Inventors: Dirk Grimm, Heidelberg (DE); Julia Fakhiri, Heidelberg (DE)

(21) Appl. No.: 16/094,788

(22) PCT Filed: Apr. 18, 2017
(86) PCT No.: PCT/EP2017/059187

§ 371 Date: Oct. 18, 2018

(30) Foreign Application Priority Data
Apr. 18, 2016 (EP) 16165783.8

(51) Int. Cl. C12N 15/113; C12N 15/66; C12N 15/86

(52) U.S. Cl. CPC C12N 15/113; C12N 2310/20; C12N 15/86; C12N 15/66

(57) ABSTRACT

The present invention relates to a method for inactivating a therapeutic polynucleotide in a host cell, comprising (a) contacting said host cell with a clustered regularly interspaced short palindromic repeats (CRISPR) RNA (gRNA) specifically hybridizing to said therapeutic polynucleotide and with a CRISPR-associated endonuclease, and, thereby, (b) inactivating said therapeutic polynucleotide. Moreover, the present invention relates to a targeting polynucleotide comprising expressible polynucleotide sequences encoding (i) a gRNA comprising a first targeting sequence specifically hybridizing to a first target sequence of interest, and, (ii) optionally, a CRISPR-associated endonuclease; wherein said targeting polynucleotide further comprises at least one inactivation sequence positioned such that such said targeting polynucleotide is inactivated by a CRISPR-associated endonuclease activity, wherein said inactivation sequence is identical to said first target sequence or is a second target sequence being non-identical to said first target sequence, preferably wherein said target sequence is identical to said first target sequence. The present invention further relates to kits, vectors, and host cells comprising said targeting polynucleotides and to the medical use of said targeting polynucleotides.

The present invention relates to a method for inactivating a therapeutic polynucleotide in a host cell, comprising (a) contacting said host cell with a clustered regularly interspaced short palindromic repeats (CRISPR) RNA (gRNA) specifically hybridizing to said therapeutic polynucleotide and with a CRISPR-associated endonuclease, and, thereby, (b) inactivating said therapeutic polynucleotide. Moreover, the present invention relates to a targeting polynucleotide comprising expressible polynucleotide sequences encoding (i) a gRNA comprising a first targeting sequence specifically hybridizing to a first target sequence of interest, and, (ii) optionally, a CRISPR-associated endonuclease; wherein said targeting polynucleotide further comprises at least one inactivation sequence positioned such that such said targeting polynucleotide is inactivated by a CRISPR-associated endonuclease activity, wherein said inactivation sequence is identical to said first target sequence or is a second target sequence being non-identical to said first target sequence, preferably wherein said target sequence is identical to said first target sequence. The present invention further relates to kits, vectors, and host cells comprising said targeting polynucleotides and to the medical use of said targeting polynucleotides.

Methods involving transfer of therapeutic DNA into a cell have been standard methods for many years now and are in the process of becoming standard methods in medical treatment, e.g. in clinical gene therapy. An unresolved issue so far is that therapeutic vectors, once transiently or stably introduced into the cells of a subject, are difficult or impossible to remove. As a potential solution, human artificial chromosomes (HAC) were proposed carrying a conditionally active kinetochor (Kononenko et al. (2015), NAR, doi: 10.1093/nar/gkv124), an approach which is only feasible if the therapeutic sequence is not incorporated into the genome of the host cell. Also, inducible suicide genes were proposed to remove cells carrying the transgene, e.g. in Zhou et al. (2015), Blood 125(26):4103. However, such an approach is feasible only if the cells carrying the transgene may be lost after treatment, which is typically not the case e.g. in gene therapy. Other well-known methods relate to the use of recombinase systems like the Cre/lox system for excision of a transgene; however, these systems leave behind at least a recombination sequence; moreover, recombination sequences must be included in the transgene before it is applied.

For modifying genomes, the Cas9/CRISPR system was developed as a versatile tool (cf, e.g. Senis et al. (2014), Biotechnol. J. 9:1; Schmidt & Grimm (2015), Biotechnol. J. 10:258). The CRISPR systems known in the art comprise an RNA, known as crRNA, of which one part is complementary to a target sequence, and a second part which has a sequence recognizable by the second component of the system, the endonuclease. In some systems, e.g. the Cas9 CRISPR system, a second type of RNA, the tracrRNA, may be required, of which a first subsequence is required to bind to the endonuclease in order to activate endonuclease activity and of which a second subsequence is complementary to said part of the crRNA sequence recognizable by the endonuclease. Preferably, the tracrRNA and the crRNA are comprised in a contiguous RNA molecule, preferably with the second subsequence of the tracrRNA and the complementary sequence of the crRNA forming a stem-loop structure. In other systems, e.g. the Prevotella and Francisella (Cpf1) CRISPR system, a tracrRNA is not required and the endonuclease recognizes the RNA complementary to the target sequence via a specific recognition sequence. The CRISPR system has been used for creating insertions and/or deletions in target DNAs, for epigenetic regulation, for labeling of DNAs, and for introducing heterologous DNA by inducing homology-driven repair.

There is, thus, a need in the art for improved means and methods for manipulating polynucleotides, in particular heterologous DNA, in a cell, avoiding the drawbacks of the prior art.

Accordingly, the present invention relates to a method for inactivating a therapeutic polynucleotide in a host cell, comprising

(a) contacting said host cell with a clustered regularly interspaced short palindromic repeats (CRISPR) RNA (gRNA) specifically hybridizing to said therapeutic polynucleotide and with a CRISPR-associated endonuclease, and, thereby,

(b) inactivating said therapeutic polynucleotide.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

The method of the present invention, preferably, is an in vitro method. Moreover, it may comprise steps in addition to those explicitly mentioned above. For example, further steps may relate, e.g., to introducing a therapeutic polynucleotide and/or obtaining a host cell for step a), or screening or selecting for host cells having an inactivated therapeutic polynucleotide following step b). Moreover, one or more of said steps may be performed by automated equipment.

The term “therapeutic polynucleotide” is used in a broad sense and relates to any polynucleotide introduced into a cell or a subject, preferably into an isolated cell, for the purpose of ameliorating a disease or disorder or the symptoms accompanied therewith and/or for retaining health for at least a certain period of time. Accordingly, the term includes polynucleotides introduced into a cell or subject to induce a change in genome structure, gene expression and/or metabolism of said cell, including, e.g., preferably, introducing a gene therapy vector, as well as introducing a vector into somatic cells to induce formation of stem cells, or into stem cells, in particular pluripotent stem cells, to induce or enhance proliferation and/or differentiation. As will be understood, the therapeutic polynucleotide may be a polynucleotide as specified herein below further comprising a therapeutic gene, i.e. the therapeutic polynucleotide may be a suicide gene therapy vector as disclosed herein. Preferably, the therapeutic polynucleotide is a DNA.

Preferably, the therapeutic polynucleotide is a polynucleotide administered within the last 50 years, preferably the last 10 years, more preferably the last 5 years before the method of the present invention is applied. Preferably, the therapeutic polynucleotide is a polynucleotide comprising a viral sequence, more preferably a sequence from a virus for which integration into a host genome is a part of the life cycle or is known to occur at a medically relevant frequency. Thus, preferably, the therapeutic polynucleotide is a sequence of a retrovirus, of an adenovirus, adeno-associated virus, or the like. More preferably, the therapeutic polynucleotide is a polynucleotide comprising a vector sequence known to be maintained extrachromosomally, preferably as an episome. Preferably, a “sequence of a virus” is an incomplete genome of a virus or a variant thereof, e.g., preferably, a sequence comprising viral terminal repeats as the only viral sequences.

Accordingly, preferably, the therapeutic polynucleotide is a non-naturally occurring polynucleotide, i.e. preferably, is an artificial polynucleotide. Thus, preferably, the therapeutic polynucleotide is a recombinant polynucleotide. More preferably, the therapeutic polynucleotide is a polynucleotide comprising nucleic acid sequences originating from at least one, more preferably of at least two species different from the species of said host cell. Preferably, the therapeutic polynucleotide comprises at least 25 nucleotides of heterologous sequence, more preferably at least 50 nucleotides, still more preferably at least 100, most preferably at least 250 nucleotides of heterologous sequence, wherein the term “heterologous polynucleotide” is understood by the skilled person and relates to a polynucleotide the nucleic acid sequence of which is derived from a species non-identical to the species of the host cell carrying said polynucleotide. Preferably, the therapeutic polynucleotide is not integrated into the genome of the host cell, more preferably is present in the host cell as an episome and/or the therapeutic polynucleotide is integrated into the genome of the host cell; i.e., preferably, the therapeutic polynucleotide is covalently linked to a chromosome of said host cell, preferably is contiguous with a chromosome of said host cell.

As used herein, the term “host cell” relates to a vertebrate cell. Preferably, the cell is a mammalian cell, more preferably, a mouse, rat, cat, dog, hamster, guinea pig, sheep, goat, pig, cattle, or horse cell. Still more preferably, the host cell is a primate cell. Most preferably, the host cell is a human cell. Preferably, the host cell comprises at least one therapeutic polynucleotide. Preferably, the host cell is a cell with a doubling time of more than 30 days, more preferably more than 90 days, even more preferably more than 180 days. Most preferably, said cell is a non-regenerating cell of a subject, preferably a pancreas cell, a lung cell, a heart cell, or a nerve cell, preferably of the central nervous system. Preferably, the host cell comprises a therapeutic polynucleotide, e.g. preferably, a recombinant viral vector.

As used herein, the term “subject” relates to a vertebrate. Preferably, the subject is a mammal, more preferably, a mouse, rat, cat, dog, hamster, guinea pig, sheep, goat, pig, cattle, or horse. Still more preferably, the subject is a primate. Most preferably, the subject is a human. Preferably, the subject is afflicted with a disease caused by or associated with integration of heterologous DNA into the genome of a cell of said subject. Also preferably, the subject is a subject comprising cells comprising a therapeutic polynucleotide. More preferably, the subject received a, preferably medical, treatment comprising administration of recombinant DNA and/or RNA, or received biological material, preferably cells, to which recombinant DNA and/or RNA was administered.

The term “inactivating a therapeutic polynucleotide”, as used herein, relates to modifying a therapeutic polynucleotide such that at least one gene comprised in said therapeutic polynucleotide is not expressed any more in a host cell. Preferably, inactivating a therapeutic polynucleotide is introducing at least one insertion or deletion into said therapeutic polynucleotide. More preferably, inactivating is introducing at least one deletion of at least 10 nucleotides, preferably at least 50 nucleotides, still more preferably at least 100 nucleotides, most preferably at least 250 nucleotides of therapeutic sequence into said therapeutic polynucleotide. Most preferably, inactivating is deleting of at least 50%, more preferably at least 75%, still more preferably at least 90%, most preferably at least 95% of said therapeutic sequence. Preferably, a multitude of therapeutic polynucleotides is inactivated, preferably removed as specified above. More preferably, all therapeutic polynucleotides in a host cell are inactivated.

The term “contacting” as used in the context of the methods of the present invention is understood by the skilled person. Preferably, the term relates to bringing a compound of the present invention in physical contact with a host cell, i.e. allowing the compound and the host cell to interact. Methods for contacting the compounds of the present invention with a host cell are, in principle, known in the art. As will be understood, contacting may comprise contacting a host cell with a polynucleotide or vector encoding one or more of the components of the present invention; in such case, contacting of the host cell with the compounds of the present invention by the host cell expressing said compounds from said polynucleotide or vector. Preferably, contacting comprises contacting said host cell with a multitude of gRNAs specifically hybridizing to a multitude of non-identical regions of said therapeutic polynucleotide.

The “clustered regularly interspaced short palindromic repeats” or “CRISPR” system is known to the skilled person, as described herein above. As used herein, the term “gRNA” includes a crRNA/tracrRNA hybrid and a crRNA-tracrRNA fusion RNA of a Cas CRISPR system, as well as a guide RNA of a CPF1 CRISPR system. More preferably, the term gRNA relates to a crRNA-tracrRNA fusion RNA of a Cas CRISPR system, as well as a guide RNA of a CPF1 CRISPR system. Most preferably, term gRNA relates to a crRNA-tracrRNA fusion RNA of a Cas CRISPR system. Preferably, the gRNA comprises at least 15, preferably at least 18, more preferably at least 20 nucleotides complementary to the target sequence. As used herein, the term “complementary”, if not otherwise noted, relates to at least 90%, more preferably at least 95%, still more preferably 99% complementarity. Most preferably complementarity relates to 100% complementarity over the aforementioned number of nucleotides. Moreover, preferably, the gRNA of the present invention further comprises a nucleotide sequence mediating binding of a Cpf1 endonuclease or comprises tracrRNA sequence, preferably of a Cas CRISPR system, more preferably of a Cas9 CRISPR system. Preferably, the gRNA comprises a structure 5′-activation sequence-targeting sequence-3′, more preferably, the gRNA comprises a structure 5′-activation sequence-linker loop-targeting sequence-3′, wherein said linker loop comprises a stem-loop comprising of from 10 to 30, more preferably of from 15 to 25 base pairs.

The term “CRISPR-associated endonuclease”, as used herein, relates to an endonuclease, preferably an endo-DNase, recognizing a gRNA as specified herein, which is, in principle, known in the art. Preferably, the CRISPR-associated endonuclease is a type II CRISPR endonuclease. Preferably, the CRISPR-associated endonuclease is a CRISPR endonuclease from Prevotella and Francisella endonuclease, i.e. a Cpf1 endonuclease. More preferably, the CRISPR endonuclease is a Cas endonuclease, still more preferably is a Cas9 endonuclease, most preferably the CRISPR-associated endonuclease is a Cas9 endonuclease from Staphylococcus aureus or is a Cas9 endonuclease from Streptococcus pyogenes.

For the avoidance of doubt, the term “target sequence” relates to a sequence the CRISPR system is directed against, wherein a targeting sequence is a sequence included in the CRISPR system to specifically direct the system to the target sequence. Thus, the target sequence is a sequence comprised in the therapeutic polynucleotide, whereas the targeting sequence is a sequence comprised in a gRNA of the present invention.

Advantageously, it was found in the work underlying the present invention that the CRISPR system can be used to inactivate therapeutic sequences from a host cell by providing a targeting gRNA targeting a subsequence of the therapeutic polynucleotide. Also, it could be shown in in vivo experiments that therapeutic vectors can successfully be targeted with the proposed method.

The definitions made above apply mutatis mutandis to the following. Additional definitions and explanations made further below also apply for all embodiments described in this specification mutatis mutandis.

The present invention further relates to a targeting polynucleotide comprising expressible polynucleotide sequences encoding

(i) a gRNA comprising a first targeting sequence specifically hybridizing to a first target sequence of interest, and,

(ii) optionally, a CRISPR-associated endonuclease;

wherein said targeting polynucleotide further comprises at least one inactivation sequence positioned such that such said targeting polynucleotide is inactivated by a CRISPR-associated endonuclease activity,

wherein said inactivation sequence is identical to said first target sequence or is a second target sequence being non-identical to said first target sequence, preferably wherein said target sequence is identical to said first target sequence.

The term “targeting polynucleotide”, as used herein, relates to a polynucleotide comprising nucleic acid sequences as specified herein, having the biological activity of encoding a gRNA of the present invention. Preferably, the polynucleotide further encodes a CRISPR-associated endonuclease. In a preferred embodiment, the polynucleotide encoding a CRISPR-associated endonuclease is provided on a separate polynucleotide, preferably as a vector as specified elsewhere herein encoding said CRISPR-associated endonuclease, more preferably comprising an inducible gene encoding said CRISPR-associated endonuclease. Thus, in a preferred embodiment, the targeting polynucleotide does not comprise a nucleotide sequence encoding CRISPR-associated endonuclease. Preferably, the targeting polynucleotide comprising an expressible polynucleotide sequence encoding a gRNA further comprises a therapeutic gene; preferably, the expressible polynucleotide sequence encoding a gRNA is inducible in such case. Also preferably, in case the targeting polynucleotide comprising an expressible polynucleotide sequence encoding a gRNA further comprises a therapeutic gene, the targeting polynucleotide does not encode a polypeptide having CRISPR-associated endonuclease activity. In the polynucleotide of the present invention, at least one inactivation sequence is positioned such that said targeting polynucleotide is inactivated by a CRISPR-associated endonuclease activity. As used herein, “inactivating a targeting polynucleotide” of the present invention is inactivating an expressible polynucleotide sequence encoding a gRNA comprised in said targeting polynucleotide and/or is inactivating an expressible polynucleotide sequence encoding a CRISPR-associated endonuclease comprised in said targeting polynucleotide and/or is abolishing maintenance of said targeting polynucleotide in a cell. Thus, preferably, the inactivation sequence(s) comprised in the targeting polynucleotide is (are) positioned such that cleavage by a CRISPR-associated endonuclease abolishes maintenance of said targeting polynucleotide in a target cell, abolishes expression of said gRNA and/or abolishes expression of said CRISPR-associated endonuclease. Accordingly, preferably, the targeting polynucleotide of the present invention is a self-inactivating polynucleotide, preferably a self-inactivating vector. As will be understood, the targeting polynucleotide may further comprise a therapeutic polynucleotide, thus, the targeting polynucleotide may be a self-inactivating therapeutic vector. Preferably, expression of the gRNA and/or the CRISPR-associated endonuclease are regulatable in the targeting vector, in particular in case the targeting vector is a self-inactivating therapeutic vector. Preferably, the inactivation sequence is present in said polynucleotide at least twice, at least two of said at least two inactivation sequences encompassing, preferably flanking, said sequence encoding a gRNA or flanking said sequence encoding said CRISPR-associated endonuclease. As will be understood, the inactivating gRNA preferably is the gRNA encoded on the polynucleotide, or is a different gRNA, preferably having a different recognition sequence. Thus, the inactivation sequence is identical to said first target sequence or is a second target sequence being non-identical to said first target sequence, preferably said target sequence is identical to said first target sequence.

In a preferred embodiment, the present invention relates to a therapeutic polynucleotide comprising (i) an expressible polynucleotide sequence encoding a gRNA comprising a first targeting sequence specifically hybridizing to a first target sequence of interest, and (ii) a therapeutic polynucleotide flanked by recombination sequences.

The term “recombination sequence” is known to the skilled person and relates to a nucleotide sequence of sufficient length and homology to mediate homologous recombination between the therapeutic polynucleotide and a corresponding sequence on the chromosome of the target subject. Preferably, in case the subject is a mammal, the 5′ recombination sequence and/or the 3′ recombination sequence have a length of at least 200 bp, more preferably at least 500 bp, most preferably at least 1000 bp. Preferably, the 5′ recombination sequence and/or the 3′ recombination sequence have at least 80%, more preferably at least 90%, still more preferably at least 95%, most preferably at least 99% sequence identity to the corresponding chromosomal sequence, determined as specified elsewhere herein. As is understood by the skilled person, the therapeutic gene, if present on the chromosome of the subject, may itself be part of the recombination sequence(s). Thus, for the purposes of the present invention, the recombination sequence may overlap the sequence of the therapeutic gene; preferably, the recombination sequence extends at least 1 bp, more preferably at least 10 bp, even more preferably at least 100 bp, most preferably at least 1000 bp beyond the therapeutic nucleotide sequence.

The term “polynucleotide variant”, as used herein, relates to a variant of a polynucleotide related to herein comprising a nucleic acid sequence characterized in that the sequence can be derived from the aforementioned specific nucleic acid sequence by at least one nucleotide substitution, addition and/or deletion, wherein the polynucleotide variant shall have the biological activity as specified for the specific polynucleotide. Preferably, said polynucleotide variant is an ortholog, a paralog or another homolog of the specific polynucleotide. Also preferably, said polynucleotide variant is a naturally occurring allele of the specific polynucleotide. Polynucleotide variants also encompass polynucleotides comprising a nucleic acid sequence which is capable of hybridizing to the aforementioned specific polynucleotides, preferably, under stringent hybridization conditions. These stringent conditions are known to the skilled worker and can be found in Current Protocols in Molecular Biology, John Wiley & Sons, N.Y. (1989), 6.3.1-6.3.6. A preferred example for stringent hybridization conditions are hybridization conditions in 6× sodium chloride/sodium citrate (=SSC) at approximately 45° C., followed by one or more wash steps in 0.2×SSC, 0.1% SDS at 50 to 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1× to 5×SSC (pH 7.2). If organic solvent is present in the abovementioned buffer, for example 50% formamide, the temperature under standard conditions is approximately 42° C. The hybridization conditions for DNA:DNA hybrids are preferably for example 0.1×SSC and 20° C. to 45° C., preferably between 30° C. and 45° C. The hybridization conditions for DNA:RNA hybrids are preferably, for example, 0.1×SSC and 30° C. to 55° C., preferably between 45° C. and 55° C. The abovementioned hybridization temperatures are determined for example for a nucleic acid with approximately 100 bp (=base pairs) in length and a G+C content of 50% in the absence of formamide. The skilled worker knows how to determine the hybridization conditions required by referring to textbooks such as the textbook mentioned above, or the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford. Alternatively, polynucleotide variants are obtainable by PCR-based techniques such as mixed oligonucleotide primer-based amplification of DNA, i.e. using degenerated primers against conserved domains of a polypeptide of the present invention. Conserved domains of a polypeptide may be identified by a sequence comparison of the nucleic acid sequence of the polynucleotide or the amino acid sequence of the polypeptide of the present invention with sequences of other organisms. As a template, DNA or cDNA from bacteria, fungi, or plants preferably, from animals may be used. Further, variants include polynucleotides comprising nucleic acid sequences which are at least 70%, at least 75%, at least 80%, at least 85%, at least 90%, at least 95%, at least 98% or at least 99% identical to the specifically indicated nucleic acid sequences. Moreover, also encompassed are polynucleotides which comprise nucleic acid sequences encoding amino acid sequences which are at least 70%, at least 75%, at least 80%, at least 85%, at least 90%, at least 95%, at least 98% or at least 99% identical to the amino acid sequences specifically indicated. The percent identity values are, preferably, calculated over the entire amino acid or nucleic acid sequence region. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (J. Mol. Evolution., 25, 351-360, 1987, Higgins et al., CABIOS, 5 1989: 151-153) or the programs Gap and BestFit (Needleman and Wunsch (J. Mol. Biol. 48; 443-453 (1970)) and Smith and Waterman (Adv. Appl. Math. 2; 482-489 (1981)), which are part of the GCG software packet (Genetics Computer Group, 575 Science Drive, Madison, Wis., USA 53711 (1991)), are to be used. The sequence identity values recited above in percent (%) are to be determined, preferably, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments.

A polynucleotide comprising a fragment of any of the specifically indicated nucleic acid sequences is also encompassed as a variant polynucleotide of the present invention. The fragment shall still have the biological function as specified above. Accordingly, the RNA or polypeptide encoded may comprise or consist of the domains of the RNA or polypeptide of the present invention conferring the said biological activity. A fragment as meant herein, preferably, comprises at least 50, at least 100, at least 250 or at least 500 consecutive nucleotides of any one of the specific nucleic acid sequences or encodes an amino acid sequence comprising at least 20, at least 30, at least 50, at least 80, at least 100 or at least 150 consecutive amino acids of any one of the specific amino acid sequences.

The polynucleotides of the present invention either consist of, essentially consist of, or comprise the aforementioned nucleic acid sequences. Thus, they may contain further nucleic acid sequences as well. Specifically, the polynucleotides of the present invention may encode fusion proteins wherein one partner of the fusion protein is a polypeptide being encoded by a nucleic acid sequence recited above. Such fusion proteins may comprise as additional part polypeptides for monitoring expression (e.g., green, yellow, blue or red fluorescent proteins, alkaline phosphatase and the like) or so called “tags” which may serve as a detectable marker or as an auxiliary measure for purification purposes. Tags for the different purposes are well known in the art and are described elsewhere herein.

The polynucleotide of the present invention shall be provided, preferably, either as an isolated polynucleotide (i.e. isolated from its natural context) or in genetically modified form. The polynucleotide, preferably, is DNA, including cDNA, or RNA, more preferably is DNA. The terms DNA and RNA encompass single as well as double stranded polynucleotides. Moreover, preferably, comprised are also chemically modified polynucleotides including naturally occurring modified polynucleotides such as glycosylated or methylated polynucleotides or artificially modified ones such as biotinylated polynucleotides. Thus, in a preferred embodiment, the gRNA is provided as such and/or the CRISPR-associated endonuclease-encoding polynucleotide is an mRNA encoding said CRISPR-associated endonuclease.

Further, the present invention relates to a kit comprising a targeting polynucleotide according to the present invention and a helper polynucleotide comprising an expressible nucleotide sequence encoding a CRISPR-associated endonuclease and/or an expressible nucleotide sequence encoding a gRNA specifically hybridizing to an inactivation sequence comprised in said targeting vector.

The term “kit”, as used herein, refers to a collection of the aforementioned components. Preferably, said components are combined with additional components, preferably within an outer container. The outer container, also preferably, comprises instructions for carrying out a method of the present invention. Examples for such the components of the kit as well as methods for their use have been given in this specification. The kit, preferably, contains the aforementioned components in a ready-to-use formulation. Preferably, the kit may additionally comprise instructions, e.g., a user's manual for applying the polynucleotides with respect to the applications provided by the methods of the present invention. Details are to be found elsewhere in this specification. Additionally, such user's manual may provide instructions about correctly using the components of the kit. A user's manual may be provided in paper or electronic form, e.g., stored on CD or CD ROM. The present invention also relates to the use of said kit in any of the methods according to the present invention.

The term “helper polynucleotide”, as used herein, relates to a polynucleotide comprising an expressible nucleotide sequence encoding a CRISPR-associated endonuclease and/or an expressible nucleotide sequence encoding a gRNA specifically hybridizing to an inactivation sequence comprised in a targeting vector. Preferably, the helper polynucleotide comprises further nucleotide sequences, more preferably, a nucleotide sequence encoding a further gRNA. Preferably, at least one of said gRNAs comprises a targeting sequence directed at sequences providing for maintenance of a targeting polynucleotide and/or helper polynucleotide in a cell (maintenance sequence), e.g. sequences comprising an origin of replication or sequences connecting the polynucleotide to the genome of a cell, directed at the gRNA and/or directed at the CRISPR-associated endonuclease of the targeting polynucleotide, i.e. comprises a targeting sequence inactivating, in the presence of a CRISPR-associated endonuclease, said sequence encoding said gRNA and/or said CRISPR-associated endonuclease on said targeting polynucleotide. Also preferably, the targeting polynucleotide of the kit comprises further nucleotide sequences, more preferably, a nucleotide sequence encoding a further gRNA. Preferably, said further gRNA comprises a targeting sequence causing inactivation of the gRNA of the helper polynucleotide, i.e. comprises a targeting sequence inactivating, in the presence of a CRISPR-associated endonuclease, said sequence encoding said gRNA on said helper polynucleotide.

More preferably, said helper polynucleotide further comprises (i) an expressible nucleotide sequence encoding a gRNA comprising a first targeting sequence specifically hybridizing to a first target sequence comprised in the targeting polynucleotide according to, and/or (ii) an expressible nucleotide sequence encoding a gRNA comprising a second targeting sequence specifically hybridizing to a second target sequence, wherein said second target sequence is non-identical to said first target sequence.

Based on the modules “expressible polynucleotide sequence encoding a gRNA”, “expressible polynucleotide sequence encoding a CRISPR-associated endonuclease”, and “inactivation sequence”, the additional targeting vectors, kits, uses and methods exemplarily depicted in FIG. 1 are envisaged by the present invention. It is understood by the skilled person from the description herein that inactivation sequences of FIG. 1 may also be placed such that maintenance of the polynucleotide is abolished; moreover, it is also understood that promoters different from those depicted, in particular regulatable promotors, may be used instead of the ones depicted in FIG. 1.

The present invention further relates to a targeting polynucleotide of the present invention and/or a kit of the present invention for use in medicine.

The present invention further relates to a targeting polynucleotide of the present invention and/or a kit of the present invention for inactivating a therapeutic polynucleotide in a subject.

Preferably, said inactivating a therapeutic polynucleotide is inactivating a therapeutic polynucleotide in a subject, i.e. preferably, is an in vivo method. Preferably, inactivating a therapeutic polynucleotide is reducing expression, preferably is abolishing expression of a therapeutic gene product. More preferably, inactivating a therapeutic polynucleotide is at least partially, preferably completely, removing said therapeutic polynucleotide from said host cell. More preferably, inactivating a therapeutic polynucleotide in a subject is preventive or curative treatment of said subject against at least one adverse effect caused or potentially caused by the presence of said therapeutic polynucleotide in said host cell. Still more preferably, said adverse event is an immunologic response to a gene product encoded by said therapeutic polynucleotide, including, without limitation, preferably, allergy and graft-versus-host disease; or said adverse event is modulation of metabolism of said host cell, of neighboring cells, of a tissue, of an organ, or of the subject by a gene product encoded by said therapeutic polynucleotide, e.g. by hormonal action of said gene product; or said adverse event is modulation of metabolism of said host cell making said cell prone to inappropriate proliferation, i.e. preferably, said adverse event is cancer.

The present invention also relates to a vector comprising a targeting and/or a helper polynucleotide according to the present invention; and to a host cell comprising a targeting and/or a helper polynucleotide or a vector of the present invention.

The term “vector”, preferably, encompasses phage, plasmid, viral or retroviral vectors as well artificial chromosomes, such as bacterial or yeast artificial chromosomes. The vector encompassing the polynucleotides of the present invention, preferably, further comprises selectable markers for propagation and/or selection in a host. The vector may be introduced into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerens. Alternatively, a plasmid vector may be introduced by heat shock or electroporation techniques. Should the vector be a virus, it may be packaged in vitro using an appropriate packaging cell line or using other appropriate methods prior to application to host cells. Retroviral vectors may be replication competent or replication defective. In the latter case, viral propagation generally will occur only in complementing host/cells.

More preferably, in the vector of the invention the target and/or helper polynucleotide is operatively linked to expression control sequences allowing expression in prokaryotic or eukaryotic cells or isolated fractions thereof. Expression of a polynucleotide comprises transcription of the polynucleotide, preferably into a translatable mRNA or into a gRNA. Regulatory elements ensuring expression in eukaryotic cells, preferably mammalian cells, are well known in the art. They, preferably, comprise regulatory sequences ensuring initiation of transcription and, optionally, poly-A signals ensuring termination of transcription and stabilization of the transcript. Additional regulatory elements may include transcriptional as well as translational enhancers. Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Moreover, inducible expression control sequences may be used in an expression vector encompassed by the present invention. Such inducible vectors may comprise tet or lac operator sequences or sequences inducible by heat shock or other environmental factors. Suitable expression control sequences are well known in the art. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pBluescript (Stratagene), pCDM8, pRc/CMV, pcDNA1, pcDNA3 (InVitrogene) or pSPORT1 (GIBCO BRL). Preferably, said vector is an expression vector and a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Moreover, inducible or repressible sequences mediating degradation of a gene may also be included in a vector of the present invention, e.g. an miRNA sequence causing the gRNA and or the mRNA encoding the CRISPR-associated endonuclease to be degraded.

The present invention also relates to an in vivo method inactivating a therapeutic polynucleotide in a subject comprising

(a) contacting cells of said subject comprising said therapeutic polynucleotide with a gRNA and with a CRISPR-associated endonuclease, and, thereby,

(b) inactivating said therapeutic polynucleotide in said subject.

The in vivo method of the present invention, preferably, comprises steps in addition to those explicitly mentioned above. For example, further steps may relate, e.g., to identifying a cell population comprising a therapeutic polynucleotide and/or providing one or more vector(s) encoding said gRNA and/or with a CRISPR-associated endonuclease for step a), or verifying the said therapeutic polynucleotide was removed after step b). Moreover, one or more of said steps may be performed by automated equipment.

In view of the above, the following embodiments are preferred:

1. A method for inactivating a therapeutic polynucleotide in a host cell, comprising

(a) contacting said host cell with a clustered regularly interspaced short palindromic repeats (CRISPR) RNA (gRNA) specifically hybridizing to said therapeutic polynucleotide and with a CRISPR-associated endonuclease, and, thereby,

(b) inactivating said therapeutic polynucleotide.

2. The method of embodiment 1, wherein said inactivating is introducing an insertion or deletion.

3. The method of embodiment 1 or 2, wherein said inactivating is at least partially removing said therapeutic polynucleotide.

4. The method of any one of embodiments 1 to 3, wherein said contacting comprises contacting said host cell with a multitude of gRNAs specifically hybridizing to a multitude of non-identical regions of said therapeutic polynucleotide.

5. The method of any one of embodiments 1 to 4, wherein a multitude of therapeutic polynucleotides is inactivated, preferably removed.

6. The method of any one of embodiments 1 to 5, wherein said CRISPR-associated endonuclease is a type II CRISPR endonuclease.

7. The method of any one of embodiments 1 to 6, wherein said CRISPR-associated endonuclease is a CRISPR endonuclease from Prevotella and Francisella (Cpf1) endonuclease.

8. The method of any one of embodiments 1 to 7, wherein said CRISPR-associated endonuclease is a Cas endonuclease, preferably is a Cas9 endonuclease.

9. The method of any one of embodiments 1 to 8, wherein said CRISPR-associated endonuclease is a Cas9 endonuclease from Staphylococcus aureus, preferably is a Cas9 endonuclease from Streptococcus pyogenes.

10. The method of any one of embodiments 1 to 9, wherein said gRNA comprises an activation sequence mediating binding and activation of said CRISPR-associated endonuclease and a targeting sequence.

11. The method of any one of embodiments 1 to 10, wherein said activation sequence is a sequence derived from a trans-activating CRISPR RNA (tracrRNA).

12. The method of any one of embodiments 1 to 11, wherein said gRNA comprises a structure 5′-activation sequence-targeting sequence-3′.

13. The method of any one of embodiments 1 to 12, wherein said therapeutic polynucleotide is a polynucleotide comprising a viral sequence, preferably a sequence of a retrovirus, of an adeno-associated virus, or of an adenovirus.

14. The method of any one of embodiments 1 to 13, wherein said therapeutic polynucleotide is a non-naturally occurring polynucleotide.

15. The method of any one of embodiments 1 to 14, wherein said therapeutic polynucleotide comprises nucleic acid sequences originating from at least two species different from the species of said host cell.

16. A targeting polynucleotide comprising expressible polynucleotide sequences encoding

(i) a gRNA comprising a first targeting sequence specifically hybridizing to a first target sequence of interest, and,

(ii) optionally, a CRISPR-associated endonuclease;

wherein said targeting polynucleotide further comprises at least one inactivation sequence positioned such that said targeting polynucleotide is inactivated by a CRISPR-associated endonuclease activity,

wherein said inactivation sequence is identical to said first target sequence or is a second target sequence being non-identical to said first target sequence, preferably wherein said target sequence is identical to said first target sequence.

17. The targeting polynucleotide of embodiment 16, wherein said inactivation sequence is a sequence heterologous to a target cell of interest.

18. The targeting polynucleotide of embodiment 16 or 17, wherein said targeting polynucleotide comprises at least two of said first target sequences encompassing at least part of the expressible polynucleotide sequence encoding said gRNA; and/or encompassing at least part of the expressible polynucleotide sequence encoding said CRISPR-associated endonuclease.

19. The targeting polynucleotide of any one of embodiments 16 to 18, wherein said targeting polynucleotide comprises at least two of said first target sequences encompassing at least part of the expressible polynucleotide sequence encoding said gRNA and/or encompassing at least part of the expressible polynucleotide sequence encoding said CRISPR-associated endonuclease, and further comprises at least two second target sequences encompassing at least part of the expressible polynucleotide sequence encoding said gRNA and/or encompassing at least part of the expressible polynucleotide sequence encoding said CRISPR-associated endonuclease, wherein said second target sequence is non-identical to said first target sequence.

20. The targeting polynucleotide of any one of embodiments 16 to 19, wherein said targeting polynucleotide further comprises an expressible gene of interest, preferably wherein said polynucleotide comprises at least one target sequence positioned such that cleavage by a CRISPR-associated endonuclease abolishes expression of said target gene.

21. The targeting polynucleotide of any one of embodiments 16 to 21, wherein said targeting polynucleotide comprises at least two of said target sequences encompassing at least part of said gene of interest.

22. A kit comprising a targeting polynucleotide according to any one of embodiments 16 to 20 and a helper polynucleotide comprising an expressible nucleotide sequence encoding a CRISPR-associated endonuclease and/or an expressible nucleotide sequence encoding a gRNA specifically hybridizing to an inactivation sequence comprised in said targeting vector.

23. The kit of embodiment 22, wherein said helper polynucleotide further comprises (i) an expressible nucleotide sequence encoding a gRNA comprising a first targeting sequence specifically hybridizing to a first target sequence comprised in the targeting polynucleotide according to, and/or (ii) an expressible nucleotide sequence encoding a gRNA comprising a second targeting sequence specifically hybridizing to a second target sequence, wherein said second target sequence is non-identical to said first target sequence.

24. A targeting polynucleotide of any one of embodiments 16 to 21, or the kit of embodiments 22 or 23 for use in medicine.

25. A targeting polynucleotide of any one of embodiments 16 to 21, or the kit of embodiments 22 or 23 for use in inactivating a therapeutic polynucleotide in a subject.

26. The targeting polynucleotide or the kit for use of embodiment 25, wherein said inactivating a therapeutic polynucleotide is reducing expression of a therapeutic gene product.

27. A vector comprising a targeting polynucleotide and/or a helper polynucleotide according to any one of embodiments 16 to 23.

28. A host cell comprising the targeting polynucleotide of any one of embodiments 16 to 21, the kit of embodiments 22 or 23, and/or the vector according to embodiment 27.

29. The host cell of embodiment 28, further comprising a least one therapeutic polynucleotide.

30. An in vivo method inactivating a therapeutic polynucleotide in a subject comprising

(a) contacting cells of said subject comprising said therapeutic polynucleotide with a gRNA and with a CRISPR-associated endonuclease, and, thereby,

(b) inactivating said therapeutic polynucleotide in said subject.

All references cited in this specification are herewith incorporated by reference with respect to their entire disclosure content and the disclosure content specifically mentioned in this specification.

### FIGURE LEGENDS

FIG. 1: Examples for permutations and applications of the invention, a) Top: prototype of a vector consisting of terminal repeats (TR, replication and packaging signals) flanking an expression cassette comprising promoter P and therapeutic polynucleotide X. Labels 1 and 2 designate binding sites for gRNAs 1 and 2, respectively. These binding sites can already be present in the vector as part of its natural sequence (i.e., the gRNA will be designed accordingly), or they can be added deliberately. Once transduced with this vector, the target cell can be treated at any time with a second vector (shown underneath the cell) encoding one or more gRNA(s) matching the binding sites in the original vector. This will result in cleavage, mutation/deletion and thus inactivation of the original vector. b) Similar to a), but the second vector now likewise contains binding sites for gRNA1. To prevent self-cleavage of this vector during production, Cas9 can be driven by tissue-specific promoter (e.g., TTR for the liver) and additionally be controlled via binding sites for cell-specific miRNAs (“miR”) that are expressed in the producer cells. The benefit of this strategy over the one in panel a is that both vectors will eventually be eliminated in the cell (in permutation a, the Cas9 will persist). c) Example for a case where the first vector expresses a gRNA for instance against a viral DNA (X) in the target cell. At any time, the cell is then transduced with a second vector encoding a gRNA2 against the first vector. Concurrently, the gRNA1 expressed by the first vector can be used to also cleave and inactivate the second vector (by inserting appropriate binding sites), resulting in simultaneous inactivation of both vectors. To further improve the specificity and stringency of this system, the second vector can exploit a Cas9 ortholog (“Cas9 o”) from a different bacterial species, thus ensuring that each vector will cleave the other. d) Variation of example a where multiple different vectors are present in the cell and can be inactivated, individually or simultaneously, by introducing a second vector expressing appropriate gRNAs. e) Example where a cell is transduced with a vector encoding therapeutic polynucleotide X and concurrently a gRNA against itself. However, because this vector lacks Cas9, it is initially stable. Instead, Cas9 can be introduced by a second vector or alternatively as naked RNA. The latter has the additional benefit that the Cas9 mRNA will be degraded over time; hence, the cell will eventually contain no foreign nucleic acid anymore. f) Self-inactivating (SIN) “all-in-one” vector which is directed against gene X in the cell as well as against itself. To prevent self-cleavage of this vector during production, expression of Cas9 can be controlled e.g., via tissue-specific promoters (e.g., TTR, inactive in the producer cell line HEK293T) and/or miRNAs (not shown here, but see example b). Additionally, the use of the stronger U6 promoter (as compared to H1) will ensure that gRNA2 is expressed at higher levels, which will favor the correct order of events in the cell, i.e., cleavage of gene X through gRNA2, followed by self-inactivation of the vector through gRNA1. FIGS. 1a), d), and f) schematically depict methods of embodiment 1; according to f), the promoter designated as U6 is stronger in the target cell as the promoter designated as H1, such that it is ensured that inactivation of the therapeutic polynucleotide X is favored; FIG. 1b) and FIG. 1f) indicates the effect of the targeting polynucleotide of embodiment 16, i.e. a self-inactivating targeting vector; FIG. 1c) indicates inactivation of a targeting polynucleotide by means of a helper polynucleotide; FIG. 1d) and FIG. 1e) indicate an inactivatable therapeutic polynucleotide.

FIG. 2: Proof-of-concept in cultured cells for an inactivatable vector according to the design in FIG. 1e. a) Scheme of vector design and experimental setting. b) Validation of the prediction from panel a using the Streptococcus pyogenes CRISPR system. c) Verification of the data of b) using the Staphylococcus aureus CRISPR system. d) Further verification using the Staphylococcus aureus CRISPR system and using Adeno-associated virus serotype 2 (AAV2) transduction instead of plasmid DNA transfection (as in panel c).

FIG. 3: Proof-of-concept in mouse livers for an inactivatable vector according to the design in FIG. 1e. a) Scheme of vector design and experimental setting. b) In vivo imaging of a mouse injected with the two vectors from panel a, or a control mouse that received only the luciferase vector, but not the Cre vector. c) Ratios of Firefly-to-Renilla luciferase in the two mice shown in panel b.

FIG. 4 Sequencing results validating successful inactivation of the novel vector in mouse livers in vivo. upper panel: sequences from Mouse #54 (Cre +): cl#1 (SEQ ID NO: 1), cl#3 (SEQ ID NO: 2), cl#2 (SEQ ID NO: 3), cl#5 (SEQ ID NO: 4), cl#4 (SEQ ID NO: 5), cl#6 (SEQ ID NO: 6), FLuc (SEQ ID NO: 7); lower panel: sequences from Mouse #61 (Cre −): cl#6 (SEQ ID NO: 8), cl#5 (SEQ ID NO: 9), cl#4 (SEQ ID NO: 10), cl#3 (SEQ ID NO: 11), cl#2 (SEQ ID NO: 12), cl#1 (SEQ ID NO: 13), FLuc(2) (SEQ ID NO: 14).

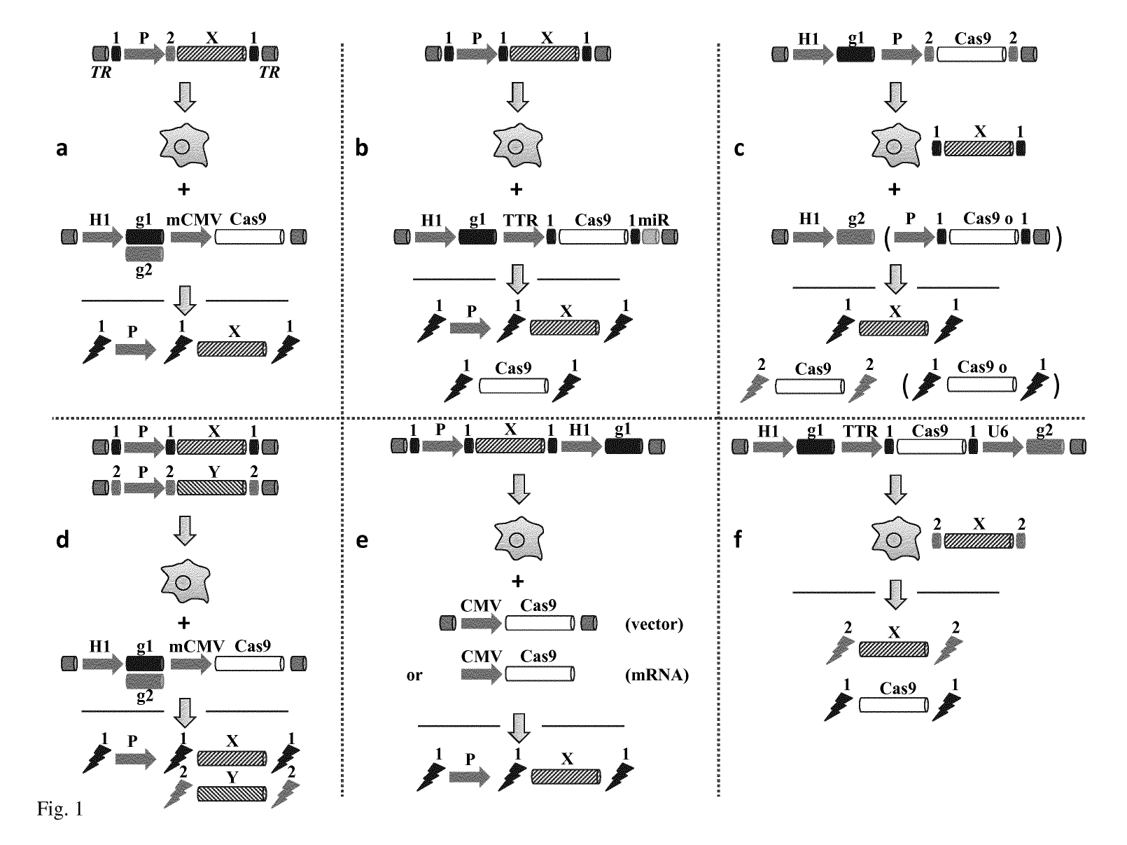

The following Examples shall merely illustrate the invention. They shall not be construed, whatsoever, to limit the scope of the invention.

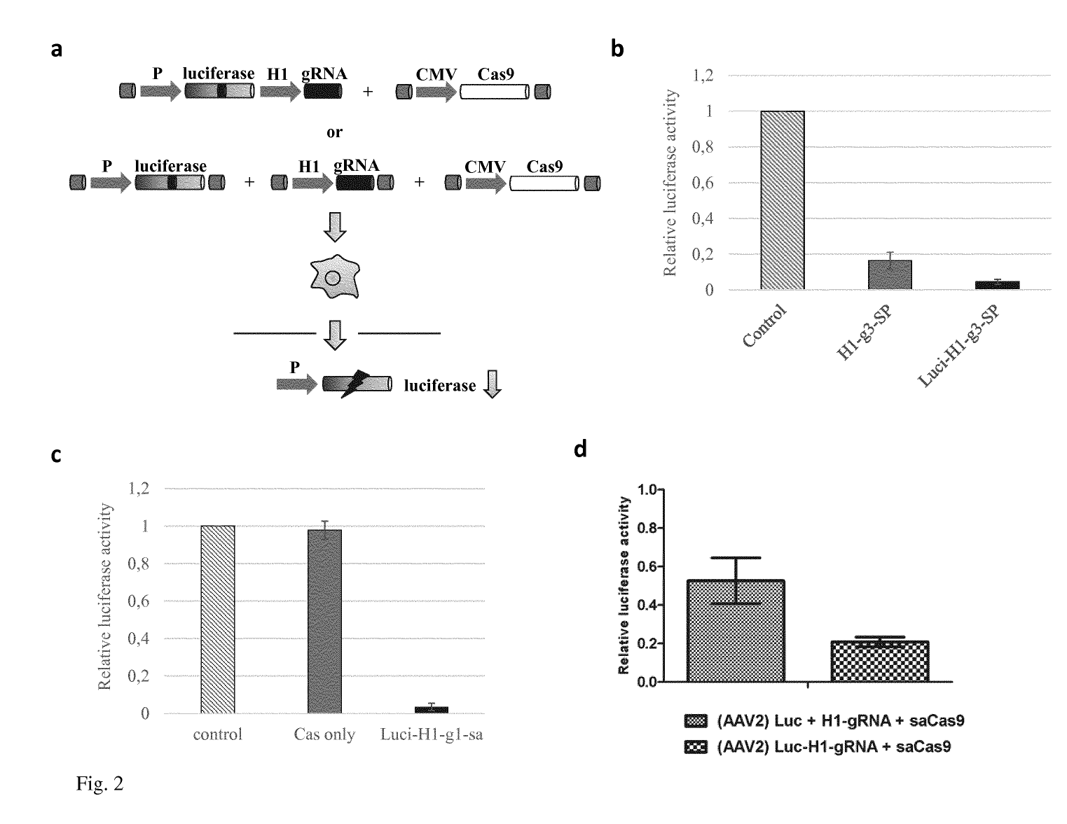

### EXAMPLE 1 (FIG. 2)

The vector used in FIG. 2 b-d is based on Adeno-associated virus (AAV) serotype 2 and expresses a Firefly luciferase gene as polynucleotide X (plus a second Renilla luciferase for normalization, not shown), together with a pre-selected potent gRNA against the Firefly luciferase gene (FIG. 2a top). Thus, in the presence of co-transfected Cas9, this vector is expected to cleave and thereby inactivate itself, resulting in a reduction of transgene (luciferase) expression.

In panel b, human embryonic kidney HEK293T cells were either co-transfected with the two vectors shown on top of panel a (lane “Luci-H1-g3-SP”), or, as control, triple-transfected with the three individual constructs also shown in FIG. 2a (bottom), i.e., one plasmid encoding the two luciferases, a second encoding the gRNA, and the third the Cas9 (bar in the middle, labeled “H1-g3-SP”). As another control, the cells were co-transfected with a construct expressing the two luciferases and an irrelevant gRNA, plus a Cas9 expression construct (negative control, bar “Control”, set to 1). Note the substantial, over 10-fold drop in the Firefly-to-Renilla luciferase ratio for the inactivatable vector (lane “Luci-H1-g3-SP”) as compared to the negative control, verifying potent self-cleavage by the encoded gRNA. This clearly illustrates the possibility to create vectors co-expressing a “suicide” gRNA directed against the vector itself and hence validates the overall concept behind the present invention.

The experiment shown in FIG. 2c) is comparable to the one in panel b), but now with gRNA and Cas9 based on or derived of, respectively, the CRISPR system from Staphylococcus aureus (the data in panel b were obtained with the Streptococcus pyogenes CRISPR system). The “Cas9 only” bar is a second negative control without any gRNA. Again, note the very potent self-cleavage and self-inhibition of the luciferase-gRNA vector in the presence of Cas9 (compare lane “Luci-H1-g1-sa” to the two controls), proving that our innovation is compatible with different CRISPR systems.

The experiment shown in FIG. 2d) is comparable to the one in panel c), but now with all components delivered by transduction with recombinant AAV2 vectors instead of plasmid DNA transfection. In sample “Luc-H1-gRNA+saCas9” (right bar), luciferase and gRNA were encoded on the same AAV vector, and Staphylococcus aureus Cas9 was delivered by a second AAV vector (i.e., setting shown on top in panel a). In sample “Luc+H1-gRNA+saCas9” (left bar), each of the three components—luciferase, gRNA or Cas9—was delivered by a separate AAV vector (i.e., setting shown at the bottom in panel a). Note that combining luciferase (target sequence) and gRNA (targeting sequence) on the same AAV vector (right bar) clearly improves the efficiency of the system, as compared to individual delivery of these two components by separate AAV vectors (left bar). These data further prove that the method described herein is compatible with different CRISPR systems, and exemplify the possibility for fine-tuning based on the configuration of the essential parts.

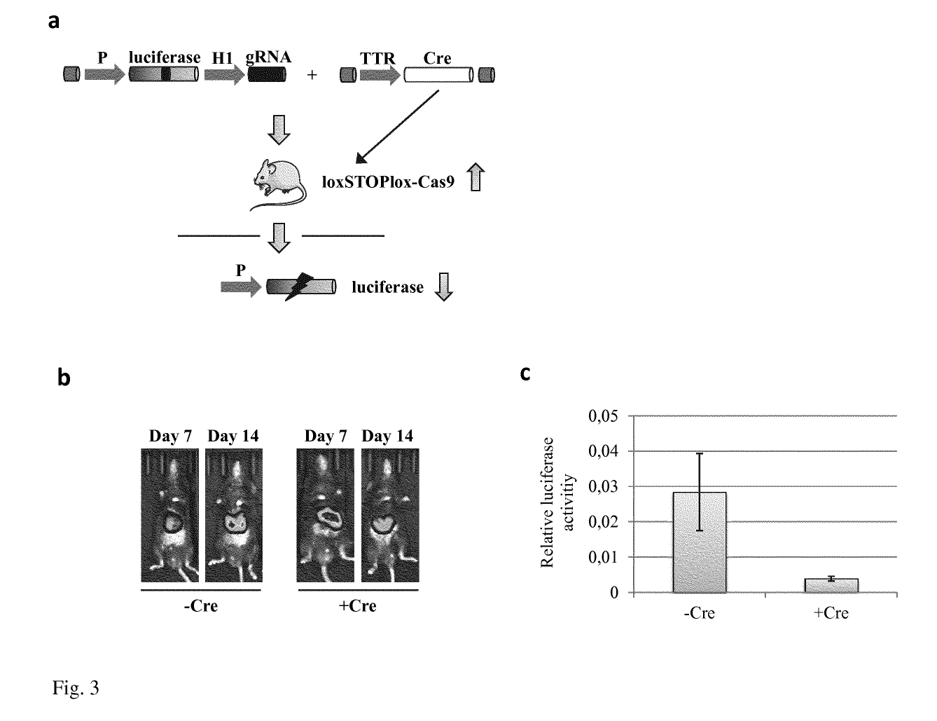

### EXAMPLE 2 (FIG. 3)

The vector is identical to the one used in the cell cultures studies in FIG. 2, i.e., it encodes a Firefly luciferase together with a gRNA against this luciferase. It was injected into mice together with a second AAV vector which expresses the Cre recombinase under the control of a liver-specific TTR (transthyretin) promoter. The mice used were transgenic and encoded the Streptococcus pyogenes Cas9 under the control of a STOP element that is flanked by loxP sites. Accordingly, it was expected that expression of the Cre recombinase from the second AAV vector would result in removal of this STOP element specifically in the liver and thus yield expression of Cas9. In turn, this should lead to self-cleavage and inactivation of the luciferase/gRNA-encoding first vector, causing a drop in Firefly luciferase levels relative to Renilla luciferase (co-encoded on the vector, not shown).

For imaging of the animals in FIG. 3b), mice were injected intraperitoneally with D-Luciferin, and photon counts were quantified 10 minutes later using a IVIS 100 camera. Note how the luciferase signal in the liver increases (darker grey tones indicate higher expression) in the mouse lacking Cre, whereas it drops in the mouse where Cre and hence Cas9 are expressed.

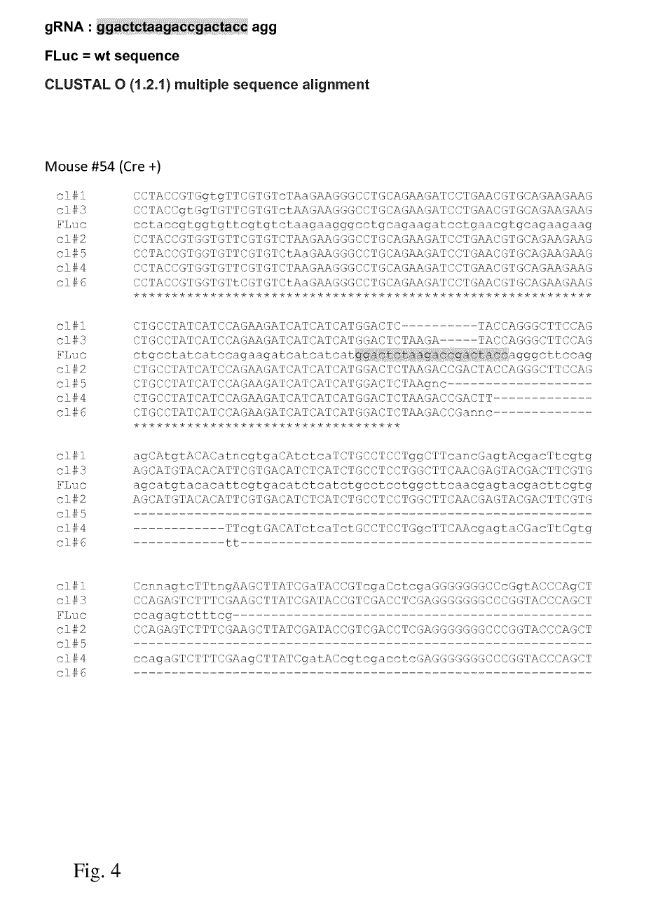

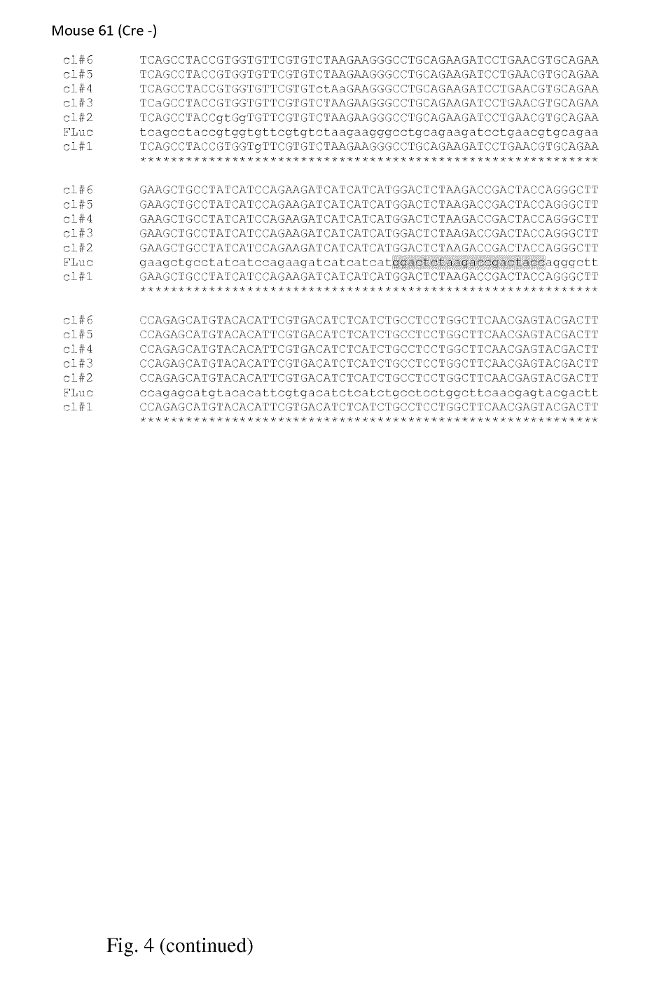

For the data of FIG. 3 c), livers were extracted 5 weeks after the initial vector injection, lysed and subjected to a Dual Luciferase Assay (Promega) (n=3). Note the lower ratios for the mouse in which Cre/Cas9 had been activated, congruent with the data in panel b and further implying successful self-inactivation of the vector in the presence of Cas9. For additional confirmation, see the sequencing data in FIG. 4.

### EXAMPLE 3 (FIG. 4)

Five weeks after vector injection, total DNA was isolated from the livers of the mice shown in FIG. 3. The target region for the anti-luciferase gRNA (highlighted in grey) was PCR-amplified and subcloned into a plasmid, and 6 randomly picked colonies from each mouse sample were sent for sequencing. The results show that in the mouse where Cre was activated (mouse #54), 5 of the 6 colonies (83.3%) showed the typical pattern of mutations that are expected for Cas9 cleavage and faulty repair by the cell. No such mutations were observed in the mouse (#61) lacking Cre/Cas9 expression, as hoped for. This complements the functional data in FIG. 3 and further verifies that CRISPR can indeed be used to target and (self-)inactivate a recombinant gene transfer vector in the liver of adult mice.

1. A method for inactivating a therapeutic polynucleotide in a host cell, comprising
(a) contacting said host cell with a clustered regularly interspaced short palindromic repeats (CRISPR) RNA (gRNA) specifically hybridizing to said therapeutic polynucleotide and with a CRISPR-associated endonuclease, and, thereby,
(b) inactivating said therapeutic polynucleotide.

2. The method of claim 1, wherein said therapeutic polynucleotide is a non-naturally occurring polynucleotide.

3. The method of claim 1, wherein said contacting comprises contacting said host cell with a multitude of gRNAs specifically hybridizing to a multitude of non-identical regions of said therapeutic polynucleotide.

4. The method of claim 1, wherein a multitude of therapeutic polynucleotides is inactivated, preferably removed.

5. The method of claim 1, wherein said CRISPR-associated endonuclease is a Cas endonuclease, preferably is a Cas9 endonuclease.

6. The method of claim 1, wherein said inactivating is at least partially removing said therapeutic polynucleotide.

7. The method of claim 1, wherein said therapeutic polynucleotide comprises nucleic acid sequences originating from at least two species different from the species of said host cell.

8. A targeting polynucleotide comprising expressible polynucleotide sequences encoding
(i) a gRNA comprising a first targeting sequence specifically hybridizing to a first target sequence of interest, and,
(ii) optionally, a CRISPR-associated endonuclease;
wherein said targeting polynucleotide further comprises at least one inactivation sequence positioned such that such said targeting polynucleotide is inactivated by a CRISPR-associated endonuclease activity,
wherein said inactivation sequence is identical to said first target sequence or is a second target sequence being non-identical to said first target sequence, preferably wherein said target sequence is identical to said first target sequence.

9. The targeting polynucleotide of claim 8, wherein said targeting polynucleotide comprises at least two of said first target sequences encompassing at least part of the expressible polynucleotide sequence encoding said gRNA; and/or encompassing at least part of the expressible polynucleotide sequence encoding said CRISPR-associated endonuclease.

10. The targeting polynucleotide of claim 8, wherein said targeting polynucleotide comprises at least two of said first target sequences encompassing at least part of the expressible polynucleotide sequence encoding said gRNA and/or encompassing at least part of the expressible polynucleotide sequence encoding said CRISPR-associated endonuclease, and further comprises at least two second target sequences encompassing at least part of the expressible polynucleotide sequence encoding said gRNA and/or encompassing at least part of the expressible polynucleotide sequence encoding said CRISPR-associated endonuclease, wherein said second target sequence is non-identical to said first target sequence.

11. A kit comprising a targeting polynucleotide according to claim 8 and a helper polynucleotide comprising an expressible nucleotide sequence encoding a CRISPR-associated endonuclease and/or an expressible nucleotide sequence encoding a gRNA specifically hybridizing to an inactivation sequence comprised in said targeting vector.

12. (canceled)

13. (canceled)

14. (canceled)

15. (canceled)

16. The method of claim 1, wherein said method comprises contacting said host cell with a targeting polynucleotide, wherein said targeting polynucleotide comprises expressible polynucleotide sequences encoding (i) a gRNA comprising a first targeting sequence specifically hybridizing to a first target sequence of interest, and, (ii) optionally, a CRISPR-associated endonuclease, wherein said targeting polynucleotide further comprises at least one inactivation sequence positioned such that such said targeting polynucleotide is inactivated by a CRISPR-associated endonuclease activity, and wherein said inactivation sequence is identical to said first target sequence or is a second target sequence being non-identical to said first target sequence, preferably wherein said target sequence is identical to said first target sequence.

17. The method of claim 1, wherein said method comprises contacting said host cell with a kit, wherein the kit comprises a targeting polynucleotide and a helper polynucleotide,
wherein the targeting polynucleotide comprises expressible polynucleotide sequences encoding
(i) a gRNA comprising a first targeting sequence specifically hybridizing to a first target sequence of interest, and,
(ii) optionally, a CRISPR-associated endonuclease,
wherein said targeting polynucleotide further comprises at least one inactivation sequence positioned such that such said targeting polynucleotide is inactivated by a CRISPR-associated endonuclease activity, and
wherein said inactivation sequence is identical to said first target sequence or is a second target sequence being non-identical to said first target sequence, preferably wherein said target sequence is identical to said first target sequence; and
wherein the helper polynucleotide comprises an expressible nucleotide sequence encoding a CRISPR-associated endonuclease and/or an expressible nucleotide sequence encoding a gRNA specifically hybridizing to an inactivation sequence comprised in said targeting vector.

18. The targeting polynucleotide of claim 8, wherein said targeting polynucleotide further comprises an expressible gene of interest, preferably wherein said polynucleotide comprises at least one target sequence positioned such that cleavage by a CRISPR-associated endonuclease abolishes expression of said target gene.

19. The kit of claim 11, wherein said helper polynucleotide further comprises (i) an expressible nucleotide sequence encoding a gRNA comprising a first targeting sequence specifically hybridizing to a first target sequence comprised in the targeting polynucleotide according to, and/or (ii) an expressible nucleotide sequence encoding a gRNA comprising a second targeting sequence specifically hybridizing to a second target sequence, wherein said second target sequence is non-identical to said first target sequence.


SEQUENCE LISTING (14 sequences)

SEQ ID NO: 1 - DNA, 230 bp, Artificial
  sequence surrounding excision site, N = any      nucleotide
  misc_feature (124)..(124): n is a, c, g, or t
  misc_feature (154)..(154): n is a, c, g, or t
  misc_feature (173)..(174): n is a, c, g, or t
  misc_feature (182)..(182): n is a, c, g, or t
cctaccgtggtgttcgtgtctaagaagggcctgcagaagatcctgaacgtgcagaagaagctgcctatcatccagaagatcatcatcatggactctaccagggcttccagagcatgtacacatncgtgacatctcatctgcctcctggcttcancgagtacgacttcgtgccnnagtctttngaagcttatcgataccgtcgacctcgagggggggcccggtacccagct

SEQ ID NO: 2 - DNA, 235 bp, Artificial
  sequence surrounding excision site, N = any      nucleotide
cctaccgtggtgttcgtgtctaagaagggcctgcagaagatcctgaacgtgcagaagaagctgcctatcatccagaagatcatcatcatggactctaagataccagggcttccagagcatgtacacattcgtgacatctcatctgcctcctggcttcaacgagtacgacttcgtgccagagtctttcgaagcttatcgataccgtcgacctcgagggggggcccggtacccagct

SEQ ID NO: 3 - DNA, 240 bp, Artificial
  sequence surrounding excision site, N = any      nucleotide
cctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgaagcttatcgataccgtcgacctcgagggggggcccggtacccagct

SEQ ID NO: 4 - DNA, 101 bp, Artificial
  sequence surrounding excision site, N = any      nucleotide
  misc_feature (100)..(100): n is a, c, g, or t
cctaccgtggtgttcgtgtctaagaagggcctgcagaagatcctgaacgtgcagaagaagctgcctatcatccagaagatcatcatcatggactctaagnc

SEQ ID NO: 5 - DNA, 215 bp, Artificial
  sequence surrounding excision site, N = any      nucleotide
cctaccgtggtgttcgtgtctaagaagggcctgcagaagatcctgaacgtgcagaagaagctgcctatcatccagaagatcatcatcatggactctaagaccgacttttcgtgacatctcatctgcctcctggcttcaacgagtacgacttcgtgccagagtctttcgaagcttatcgataccgtcgacctcgagggggggcccggtacccagct

SEQ ID NO: 6 - DNA, 109 bp, Artificial
  sequence surrounding excision site, N = any      nucleotide
  misc_feature (105)..(106): n is a, c, g, or t
cctaccgtggtgttcgtgtctaagaagggcctgcagaagatcctgaacgtgcagaagaagctgcctatcatccagaagatcatcatcatggactctaagaccgannctt

SEQ ID NO: 7 - DNA, 193 bp, Artificial
  sequence of FLuc construct
cctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcg

SEQ ID NO: 8 - DNA, 180 bp, Artificial
  sequence surrounding excision site, N = any      nucleotide
tcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgactt

SEQ ID NO: 9 - DNA, 180 bp, Artificial; identical to SEQ ID NO: 8
  sequence surrounding excision site, N = any      nucleotide

SEQ ID NO: 10 - DNA, 180 bp, Artificial; identical to SEQ ID NO: 8
  sequence surrounding excision site, N = any      nucleotide

SEQ ID NO: 11 - DNA, 180 bp, Artificial; identical to SEQ ID NO: 8
  sequence surrounding excision site, N = any      nucleotide

SEQ ID NO: 12 - DNA, 180 bp, Artificial; identical to SEQ ID NO: 8
  sequence surrounding excision site, N = any      nucleotide

SEQ ID NO: 13 - DNA, 180 bp, Artificial; identical to SEQ ID NO: 8
  sequence surrounding excision site, N = any      nucleotide

SEQ ID NO: 14 - DNA, 180 bp, Artificial; identical to SEQ ID NO: 8
  second Fluc sequence